(19) United States
(12) Patent Application Publication
(10) Pub. No.: US 2004/0167473 A1
(43) Pub. Date: Aug. 26, 2004

(54) Trocar-cannula complex, cannula and method for delivering fluids during minimally invasive surgery

(76) Inventor: Stephen P. Moenning, Punta Gorda, FL (US)

Correspondence Address: WOOD, HERRON & EVANS, LLP, 2700 CAREW TOWER, 441 VINE STREET, CINCINNATI, OH, 45202, US

(21) Appl. No.: 10/786,647

(22) Filed: Feb. 25, 2004

Related U.S. Application Data
(63) Continuation of application No. PCT/US02/29356, filed on Sep. 17, 2002.
(63) Continuation-in-part of application No. 09/934,399, filed on Aug. 21, 2001, now Pat. No. 6,695,815.
Application No. 09/934,399 is a continuation of application No. 09/511,100, filed on Feb. 23, 2000, now Pat. No. 6,302,873.
(60) Provisional application No. 60/325,806, filed on Sep. 28, 2001.
(60) Provisional application No. 60/341,032, filed on Dec. 12, 2001.

(51) Int. Cl. A61M 5/178

(52) U.S. Cl. 604/164.02; 604/247

(57) ABSTRACT

A fluid delivery cannula which provides an interface between an access point or port in the body of a patient and a working channel which may receive tools or instruments used during minimally invasive surgery. The cannula allows introduction of fluid(s) at the port site, or another site within the body of the patient. The fluid delivery cannula can releasably attach to a hub associated with a trocar assembly or may be integrally formed with at least a portion of the hub. In one form, an inner cannula member includes a grooved outer surface to define multiple fluid passages and a perforated outer layer is placed over the inner cannula member. Another form provides an expandable sleeve that may itself comprise a cannula through which a trocar or trocar assembly is inserted or which may take the place of the perforated outer layer of the grooved cannula.

The present application is a continuation of PCT Serial No. PCT/US02/29356 filed on Sep. 17, 2002, now pending, which claims the priority of U.S. Provisional Patent Application Serial No. 60/325,806, filed on Sep. 28, 2001 (now abandoned) and 60/341,032, filed on Dec. 12, 2001 (now abandoned), and which is a continuation-in-part of U.S. Ser. No. 09/934,399, filed on Aug. 21, 2001 (now U.S. Pat. No. 6,695,815) which is a continuation of U.S. Ser. No. 09/511,100 filed on Feb. 23, 2000 (now U.S. Pat. No. 6,302,873). The disclosures of each of these prior related applications are hereby fully incorporated by reference herein.

### FIELD OF THE INVENTION

This invention generally relates to cannulas and, more specifically, to cannulas used during minimally invasive surgery for allowing the introduction of instruments, such as laparoscopic tools, during surgical procedures.

### BACKGROUND OF THE INVENTION

Minimally invasive surgery is a popular alternative to more traditional surgery. This is due to the fact that minimally invasive surgery generally results in less pain and shorter hospital stays for the patient. Also, the cost of performing a surgical procedure through minimally invasive techniques can be substantially less than more traditional surgical approaches.

Minimally invasive surgical techniques require access into the body of a patient through a small working channel of an apparatus known as a trocar-cannula complex. A relatively small access incision is made in the patient at the appropriate location on the patient to receive the trocar-cannula complex. When the trocar-cannula complex is combined with long, narrow instruments, the resulting assembly allows a surgeon to work inside the body through the small access incision or port site. This approach has resulted in the aforementioned clinical advantages and extensive health care cost savings.

Traditionally, the trocar-cannula complex has been configured with three parts. The first part is the top portion and is referred to in the medical industry as the hub. The hub defines the entrance to the trocar-cannula complex and also includes various seals and air insufflation components. The second part is the trocar, which is a long, narrow blade extendable through an inner cannula to allow smooth penetration into the body of the patient through the tissue layers. The third portion is an outer cannula which is a tubular member of the complex adapted to pass into the body cavity. The outer cannula provides an interface between the patient's tissue at the access incision or port site and the trocar assembly.

Minimally invasive surgery has grown in popularity in recent years and many new types of trocar-cannula products have been proposed and introduced to address different surgical needs and procedures. The various trocar-cannula complexes include reusable and disposable cannulas and trocars, as well as hybrid varieties that comprise combinations of reusable and disposable components of the trocar-cannula complexes. A complex which is a combination of reusable and disposable components is known as a resposable device. Such devices continue to improve surgical outcomes and economics.

Animal studies on cancer treatments involving the performance of minimally invasive surgery point to a growing body of evidence which supports the concept of delivering an irrigant to the port site after the surgical procedure. In these studies, the irrigants were delivered by a syringe and needle and included substances such as betadine, saline and lidocaine. These studies showed that irrigating the port site with such substances immediately after the surgical procedure beneficially resulted in a lower incidence of infection or less pain, depending on the irrigant. However, the technique also resulted in increased operative time and increased exposure of the surgical staff to needle sticks. In addition, the potential for contaminants to spread to the port site during the surgery has been well documented. Irrigation performed only at the end of the surgical procedure unfortunately cannot reduce patient exposure to contaminants during the procedure.

In view of the above-mentioned drawbacks in the field, there is a need for more effective delivery of fluids to an access point or port in the body of a patient before, during, and/or after the performance of minimally invasive surgery. Such delivery of fluid(s) could assist in patient treatment, such as through the delivery of cancer treatment medication or other medication, as well as reduction of port site contamination and infection, and reduction of post-operative pain. Other uses of the invention may be made in connection with delivering any desired fluid to a patient.

### SUMMARY OF THE INVENTION

The present invention generally relates to a unique fluid delivery cannula which provides an interface between an access point or port in the body of a patient and a working channel which may receive tools or instruments used during minimally invasive surgery. In accordance with one general aspect of the invention, the cannula allows introduction of fluid(s) at the port site, or another site within the body of the patient, at any time after the cannula is introduced through the access point or port site of the body. The fluids may be introduced manually, such as through a manually operated syringe coupled in fluid communication with one or more fluid passages in or on the wall of the cannula. Alternatively, the fluids may be delivered automatically through a suitable medical pump or other device.

The fluids may include, for example, saline solution, lidocaine-containing fluids, betadine-containing fluids, or other substances, depending on the intended use and desired purpose. Presently, it is contemplated that such fluids will be especially beneficial to reduce post-operative pain, prevent infection and contamination at the port site and provide for many types of treatment to an affected area within the body of the patient. Another potential use is for delivering tissue adhesive to the patient.

In one embodiment, the fluid delivery cannula releasably attaches to the hub. In another embodiment, the fluid delivery cannula is integrally formed with at least a portion of the hub. As one example, the fluid delivery cannula may be integrally molded with a housing portion which is configured to receive valving components and/or other insufflation components, while also allowing the trocar to pass through into the fluid delivery cannula. The hub can include a fluid inlet comprising a coupling, such as a standard luer connection, for receiving a manually operated syringe device allowing for the injection of the desired fluids. The fluid delivery cannula preferably has, in addition to a main lumen for receiving the trocar, one or more fluid passages for irrigation purposes. In the preferred embodiments, the cannula has a layered construction with multiple fluid passages contained between two layers of the cannula. The outside surface of one layer of the cannula includes grooves or recesses in fluid communication with the fluid inlet and an outer layer of the cannula includes one or more apertures or perforations communicating with the grooves for dispensing the fluid. Also in the preferred embodiment, the outside portion of the cannula, which has the fluid dispensing apertures, provides a visual target zone for the accurate delivery of the fluid to the port site. For example, this may comprise using a different color, texture, or other visually identifiable indicia at that fluid dispensing location of the cannula such that the surgeon can accurately determine where the fluid is being directed.

The invention may be manufactured in many different manners while still functioning in accordance with the inventive principles. As mentioned above, the preferred form of the invention includes an inner cannula member having a grooved outer surface to define multiple fluid passages. An outer layer of biocompatible material (e.g., PTFE) is preferably heat shrunk onto the outer surface to enclose and seal the grooves to form passages. This biocompatible material includes, preferably, several apertures positioned around the circumference of the cannula and communicating with the grooves so that the fluid may be dispensed around the entire circumference of the cannula at a specific location along the length thereof. Alternatively, or in addition, fluid passages and one or more apertures may be provided only at one location about the circumference of the cannula for even more targeted delivery of the fluid.

As alternative embodiments, the outer layer may be comprised of a layer which is configured similar to a condom and rolled onto the cannula and which includes the necessary aperture(s) for fluid delivery to the patient. The outer layer may be a rigid layer which is coupled to the inner cannula member in a rigid fashion or, for example, in a movable fashion such as a rotatable fashion to allow opening, closing, or size adjustment of the fluid delivery passage(s). As one additional alternative, the outer layer may be formed at least partially of a porous material which provides the necessary apertures. Such porous materials may, for example, take the form of sintered metals, filter media, paper, mesh cloth or a porous plastic.

Another embodiment of the invention provides an expandable sleeve that may itself comprise a cannula through which a trocar or trocar assembly is inserted or which may take the place of the perforated outer layer of the grooved cannula discussed above. Other expandable sleeve embodiments may also be configured in accordance with this aspect of the invention as well. Such an expandable sleeve can, for example, allow trocars having different diameters to be inserted through the sleeve. Therefore, the same expandable fluid delivery sleeve may be used in connection with different sized trocars or trocar assemblies thereby reducing or eliminating the need for different sized fluid delivery cannulas or sleeves.

Various objects, advantages and features of the invention will become more readily apparent to those of ordinary skill upon review of the following detailed description of the preferred embodiment taken in conjunction with the accompanying drawings.

### BRIEF DESCRIPTION OF THE DRAWINGS

FIG. 1 is a perspective view showing a trocar-fluid delivery cannula complex constructed in accordance with the invention and being used during a minimally invasive surgical procedure.

FIG. 2 is a cross sectional view taken generally along the longitudinal axis of the trocar-fluid delivery cannula complex of FIG. 1 for showing the irrigant flow path.

FIG. 3 is an enlarged cross sectional view similar to FIG. 2, but more clearly showing the flow path for the delivery of fluid through the cannula.

FIG. 4 is a cross sectional view taken along line 4-4 of FIG. 2.

FIG. 5 is a plan view of the fluid delivery cannula with the outer layer or sheath removed for clarity.

FIG. 6 is a plan view of another embodiment in which the fluid delivery cannula is integrally formed with a portion of a trocar hub.

FIG. 7 is a cross sectional view taken along line 7-7 of FIG. 6.

FIG. 8 is a longitudinal cross sectional view similar to FIG. 2, but illustrating an alternative embodiment of the invention incorporating an expandable fluid delivery sleeve.

FIG. 9 is a perspective view of another alternative embodiment of an expandable fluid delivery sleeve or cannula.

FIG. 10 is a cross sectional view taken along line 10-10 of FIG. 9.

FIG. 11 is an enlarged perspective view of the distal end of another expandable fluid delivery sleeve or cannula.

### DETAILED DESCRIPTION OF THE PREFERRED EMBODIMENT

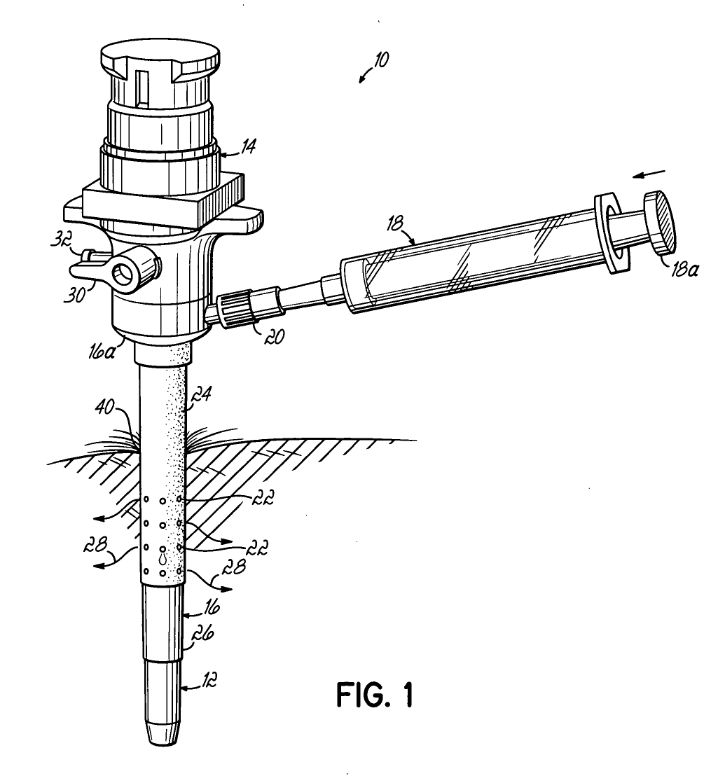

FIG. 1 illustrates a trocar-fluid delivery cannula complex 10 constructed in accordance with one preferred embodiment of the invention. Complex 10 includes a trocar assembly 12 which may include a conventional hub assembly 14. Representative trocar assemblies are shown and described in previous patents, such as my previous U.S. Pat. Nos. 6,063,060; 6,039,725; 5,865,817; and 5,865,809, the disclosures of which are hereby fully incorporated by reference herein. In accordance with the invention, a cannula 16 is positioned on the outside of trocar assembly 12 and includes a base portion 16a. A syringe 18 couples to base portion 16a of cannula 16 through a fluid coupling, such as a standard luer connector assembly 20. A plunger 18a of syringe 18 is used to manually inject a fluid into base portion 16a of cannula 16. An outer layer or sheath 24, preferably formed of PTFE (Teflon®), is secured to the outer surface of an inner tube 26 of cannula 16 and includes apertures 22. In the preferred embodiment, sheath 24 is a tube which is heat shrunk onto inner tube 26 but it may take other forms and may be secured in other ways. As will be described below, cannula 16 includes appropriate fluid passages communicating with an inlet passage in base portion 16a to allow the fluid to be dispensed through apertures 22 as shown by arrows 28. Hub assembly 14 further includes an insufflation valve 30 and a gas inlet 32 for receiving a pressurized gas, such as CO₂.

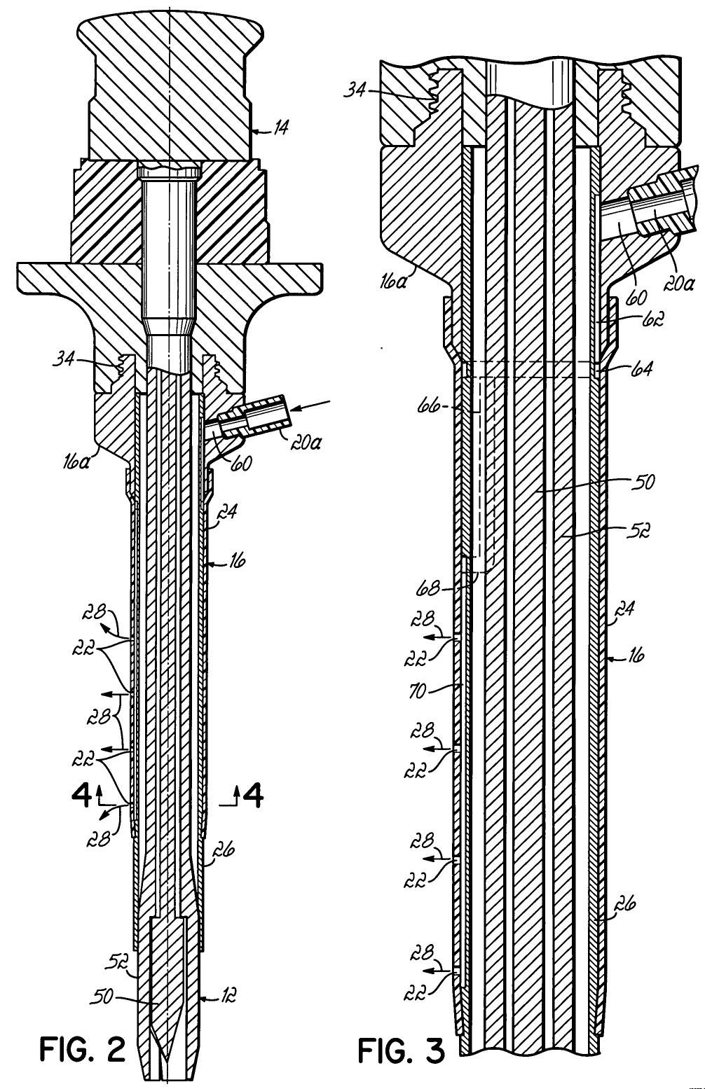

As further shown in FIGS. 2 and 3, base portion 16a of cannula 16 is threaded onto hub assembly 14 by threads 34. Thus, cannula 16 may be easily coupled to and decoupled from hub assembly 14. In the preferred embodiment, cannula 16 is disposable, however, it also may be manufactured as a reusable device intended to be sterilized between uses. Trocar assembly 12 more specifically comprises a trocar 50 received by a protective shield 52. It will be appreciated that other instruments and tools may be inserted through the working channels formed by either irrigating cannula 16 or other tubular member(s) positioned within cannula 16. This includes many other configurations of trocars or trocar assemblies as generally recognized in the art.

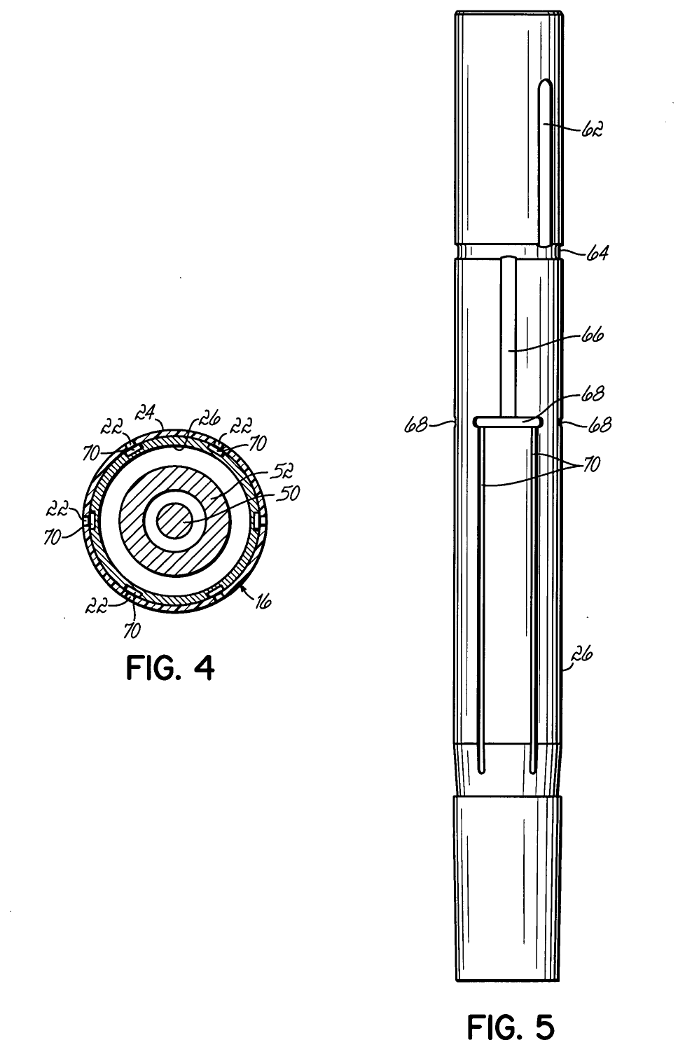

More specifically referring to FIGS. 3-5, irrigation fluids are introduced through luer connector 20a (FIG. 3) into fluid inlet 60 and groove or channel 62 formed in inner tube 26 of cannula 16. Groove 62 communicates with an annular, circumferential groove 64 and groove 64 communicates with three separate longitudinal grooves 66 which are spaced in 120° increments about inner tube 26. Grooves 66 respectively communicate with three partially annular grooves 68 which, in turn, each communicate with two longitudinal grooves 70. Longitudinal grooves 70 communicate with apertures 22 in sheath 24 and apertures 22 thereby dispense the fluid at the port site 40 or, if cannula 16 is appropriately inserted and positioned, elsewhere within the patient.

As mentioned above, the outer sheath 24 of the cannula 16 is preferably formed of PTFE and, more preferably, the outer sheath 24 is transparent or at least translucent. In addition, the area of sheath 24 containing apertures 22 may be formed with a distinct color, texture or other visually identifiable indicia which allows the surgeon to accurately position the apertures 22 with respect to the tissue to be infused with irrigation fluid. The various grooves in the outside surface of the inner tube 26 may be substituted with one or more passages within the walls of the inner tube 26 and may be of any suitable configuration and shape so long as the function of delivering fluid through the wall of the cannula 16 is facilitated by the configuration. The outer wall or sheath is a heat shrinkable material, such as an elastomeric material, however, this may also be substituted by other components or even eliminated, for example, if the passages and apertures are in the wall of an integrally formed cannula or if another fluid delivery structure is carried on the outer cannula. The inner tube in the preferred embodiment is preferably formed from aluminum with the various grooves in its outer surface being machined, however, it may instead be formed of other materials, such as plastic materials, and formed by other techniques such as molding. The preferred embodiment is especially advantageous in that it is simple to manufacture and the outer sheath forms a seal at the upper and lower ends of the inner tube while, at the same time, defining walls of the internal passages formed by the various grooves.

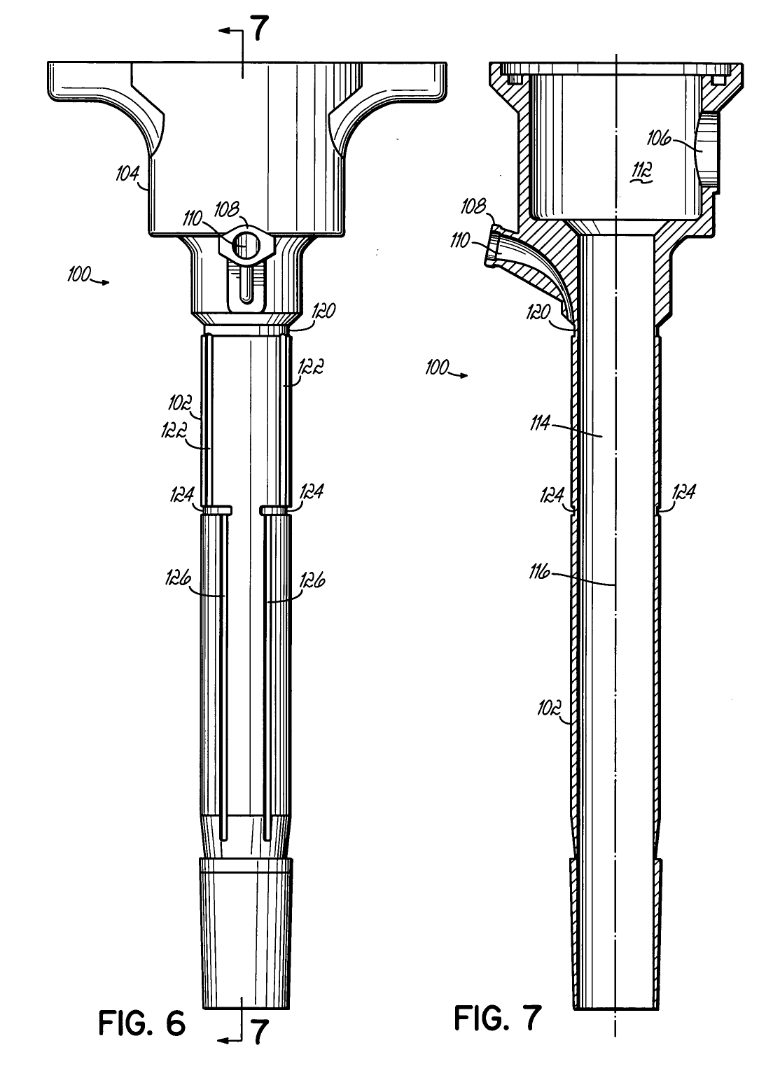

FIGS. 6 and 7 illustrate a second illustrative embodiment of the invention comprising an fluid delivery cannula 100 which includes an irrigating portion 102 and a hub or housing portion 104 formed in one piece. For example, the entire structure shown in FIGS. 6 and 7 may be molded from a polymeric material, such as conventional medical grade polymers, using Mu-cell technology or other appropriate molding techniques. In FIGS. 6 and 7, the outer layer or sheath containing the one or more perforations has been removed for clarity. Housing portion 104 includes a port 106 for receiving valving and gas input components as are known in the art. A fluid input 108 is formed on cannula 100 and communicates with a passage 110 for the introduction of the necessary or desired fluids to irrigation portion 102. A space 112 is provided for the necessary valving, sealing components, etc., typically used in trocar hubs. A lumen 114 extends along an axis 116 for receiving the trocar (not shown) and other working instruments. A system of fluid delivery passages is formed on the outside surface of irrigation portion 102 in the same illustrative pattern as discussed with respect to the first embodiment. This includes an annular groove 120 which communicates with passage 110 and delivers the fluid to three separate longitudinal passages 122 positioned at 120° increments around the outside surface of irrigation portion 102 relative to axis 116. Grooves 122 communicate with respective partially annular grooves 124. Again, while only two grooves 124 are shown in the drawings, a total of three grooves are formed in the outer surface of irrigation portion 102 positioned at 120° increments about axis 116. Each partially annular groove 124 communicates with two separate longitudinal grooves 126. Although only two grooves 126 are shown in FIG. 6, it will be appreciated that a total of six such grooves are formed in the outer surface of irrigation portion 102 in this particular embodiment. As in the first embodiment, grooves 126 communicate the fluid to perforations in the outer sheath (not shown) which then deliver the fluid to the patient. The outer sheath, as in the first embodiment, is preferably heat shrunk onto irrigation portion 102 so as to seal all of the grooves in the same manner as shown, for example, in FIGS. 2 and 3 of the first embodiment. As mentioned above, it will be appreciated that many other configurations of fluid delivery passages may be utilized in the cannula within the spirit and scope of this invention.

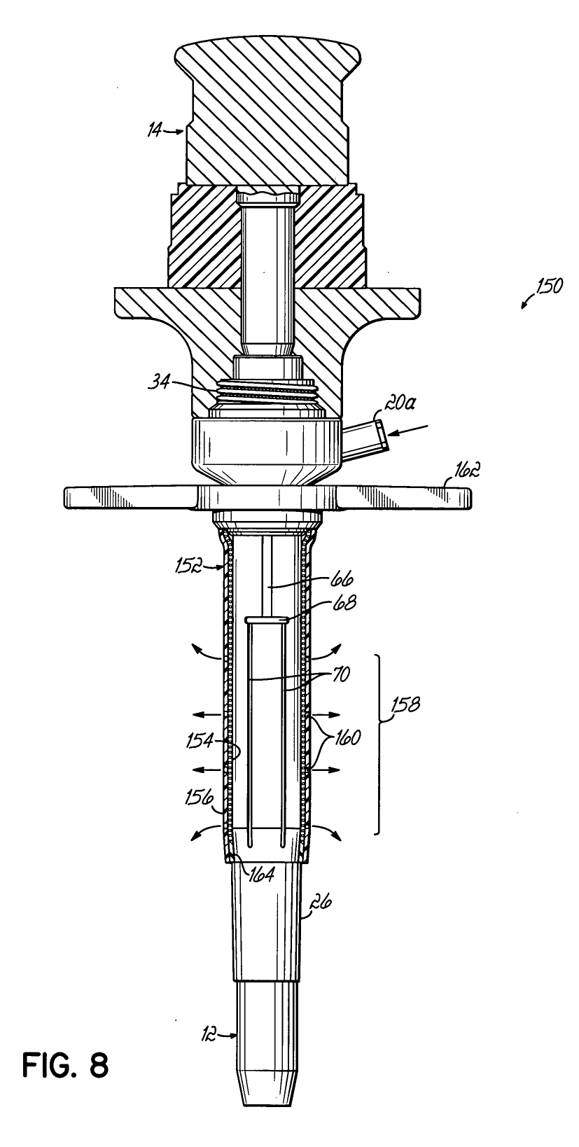

In FIG. 8, like reference numerals refer to like elements of structure between the two embodiments. In the alternative trocar-cannula complex 150 of FIG. 8, the outer sleeve or layer 24 (not shown) which was affixed to the grooved cannula 26 has been removed and replaced by an expandable sleeve 152. Expandable sleeve 152 may be a layered construction including a mesh layer 154 and an outer elastomeric layer 156. Layer 156 is uniformly perforated about its entire periphery, such as in a circumferential zone 158 as shown in FIG. 8, so that at least some of the perforations 160 line up with the longitudinal grooves 70 of the cannula 26. Thus, fluid is delivered through input 20a and into grooves 66, 68, 70 as described previously with respect to the first embodiment and this fluid is transferred through the expandable inner mesh layer 154 and expandable outer elastomeric layer 156 containing perforations 160. It will be appreciated that many other forms than the layered mesh construction shown may be used in place of the expandable sleeve 152 shown in FIG. 8. FIG. 8 illustrates the use of the expandable sleeve 152 in connection with a 10 mm trocar assembly, however, in accordance with this aspect of the invention, the expandable fluid delivery sleeve 152 may alternatively be used with other trocars having larger or smaller diameters. A rigid handle portion 162 is provided at the proximal end of sleeve 152 to allow application and removal of sleeve 152 to and from trocar 12. In order to seal the distal end of the expandable sleeve, a seal 164 may be provided distally of the mesh layer 154 as generally illustrated in FIG. 8. Alternatively, this seal 164 may be eliminated and the mesh layer 154 could then allow additional fluid to be delivered from the distal end of the sleeve 152.

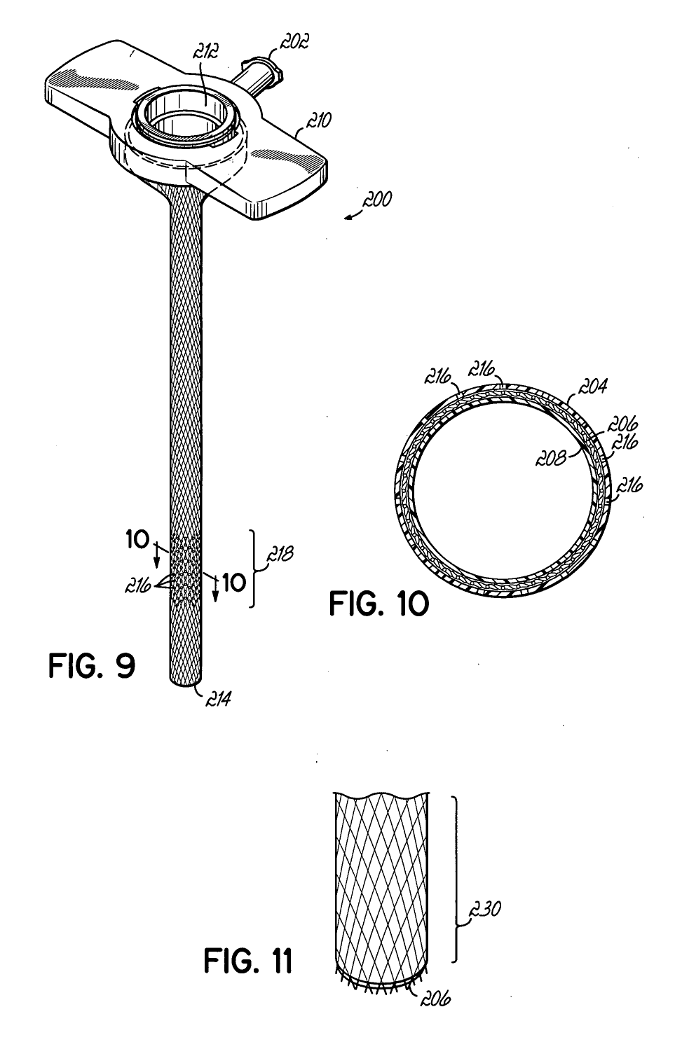

FIGS. 9 and 10 illustrate another embodiment of an expandable fluid delivery sleeve 200 which does not need the separate cannula 26 (FIG. 8) for fluid delivery as in the embodiment of FIG. 8. Instead, this sleeve 200 is formed in a manner allowing fluid delivery to take place via an input 202 and sleeve 200 alone. Sleeve 200 is formed of a layered construction including an outer perforated layer 204, an intermediate mesh layer 206, and an inner layer 208. Each layer 204, 206, 208 is expandable such that sleeve 200 may be used effectively on trocars having different diameters. The intermediate mesh layer 206 allows fluid to travel through the interstices therein from an appropriate fluid passageway extending through input 202 and an upper handle portion 210. Alternatively, other types of fluid passages may be utilized. A trocar (not shown) is inserted through the bore 212 at the proximal end such that it extends through the distal end 214 of the expandable sleeve 200. Perforations 216 are preferably formed in a desired zone 218 of sleeve 200 generally as described with respect to the previous embodiments. This zone 218 may be formed of a different color or in any other manner which indicates the positioning of the perforations to the doctor during the surgical procedure. Although not shown in FIGS. 9 and 10, this sleeve 200 may also have a seal at the distal end 214 to prevent fluid from leaking out the distal end 214.

As exemplified in FIG. 11, a distal end 230 of the expandable sleeves may be formed so as to allow fluid delivery to take place directly at the distal end. This aspect is shown in FIG. 11 schematically by indicating that the intermediate mesh layer 206 extends slightly beyond the other layers or is otherwise unsealed and, therefore, the fluid pathway through the mesh material 206 remains unblocked at the distal end 230. This general aspect of fluid delivery from the distal end 230 may be used alone or in conjunction with fluid delivery from surface perforations as previously described.

Many different types of irrigation fluids may be introduced through the fluid delivery cannulas of this invention. These include, but are not limited to, saline solutions, lidocaine-containing fluids, betadine-containing fluids, cancer treatment fluids, or any other fluid necessary or desired for a particular medical procedure. In addition, fluids other than irrigation fluids or treatment fluids may be delivered through the cannulas of this invention. As one additional example, bioadhesives may be delivered to an incision site or any other necessary tissue repair site to provide for quicker and more effective administration of the adhesive to the desired site. Many different types of trocars and cannulas may be utilized within the scope of this invention. These trocars and cannulas may be inserted through a port site of a patient together in one operation or separately, for example, by using a needle introducer for an expandable cannula and subsequently introducing the trocar.

While the present invention has been illustrated by a description of a preferred embodiment and while this embodiment has been described in some detail, it is not the intention of the Applicant to restrict or in any way limit the scope of the appended claims to such detail. Additional advantages and modifications will readily appear to those skilled in the art. The various features of the invention may be used alone or in numerous combinations depending on the needs and preferences of the user. This has been a description of the present invention, along with the preferred methods of practicing the present invention as currently known. However, the invention itself should only be defined by the appended claims, wherein I claim:

1. A trocar-cannula complex for use in minimally invasive surgical procedures performed through a port site of a patient, comprising:
a trocar; and
a fluid delivery cannula comprising a tubular structure including a central lumen receiving said trocar and an outer surface adapted to interface with tissue at the port site, said fluid delivery cannula further including at least one fluid passage having an inlet and an outlet, said outlet communicating with said outer surface for delivering fluid thereto.

2. The trocar-cannula complex of claim 1, further comprising a hub portion having valving components operative to deliver insufflation gas to the patient, said hub portion being coupled to said fluid delivery cannula in a releasable manner.

3. The trocar-cannula complex of claim 1, further comprising a hub portion having valving components operative to deliver insufflation gas to the patient, said hub portion being formed integrally with said fluid delivery cannula.

4. The trocar-cannula complex of claim 3, wherein said hub portion and said cannula are integrally molded from a polymeric material.

5. The trocar-cannula complex of claim 1, wherein said tubular structure is radially expandable.

6. A trocar-cannula complex for use in minimally invasive surgical procedures performed through a port site of a patient, comprising:
a trocar; and
a fluid delivery cannula comprising a multilayer tubular structure including a central lumen receiving said trocar and an outer surface adapted to interface with tissue at the port site, said fluid delivery cannula further including at least one fluid passage having an inlet and an outlet and being at least partially positioned between two separate layers of said tubular structure, said outlet communicating with said outer surface for delivering fluid thereto.

7. The trocar-cannula complex of claim 6, wherein said two separate layers include an inner rigid tubular member and an outer sheath carried on said inner rigid tubular member, said inner rigid tubular member including a grooved surface for providing said fluid passage and said outer sheath operative to seal said fluid passage against leakage.

8. The trocar-cannula complex of claim 7, wherein said outer sheath is comprised of a polymeric material carried on said grooved surface.

9. The trocar-cannula complex of claim 8, wherein said polymeric material includes PTFE.

10. The trocar-cannula complex of claim 8, wherein said outer sheath is heat shrunk onto said grooved outer surface.

11. The trocar-cannula complex of claim 7, wherein said outer layer is radially expandable.

12. A fluid delivery cannula for use in minimally invasive surgical procedures performed through a port site of a patient, comprising:
a tubular structure including a central lumen configured to receive a trocar and an outer surface adapted to interface with tissue at the port site, said tubular structure further including at least one fluid passage having an inlet and an outlet, said outlet communicating with said outer surface for delivering fluid thereto.

13. The fluid delivery cannula of claim 12, wherein said tubular structure is formed by multiple layers and said fluid passage is located between at least two of said layers.

14. The fluid delivery cannula of claim 12, further comprising a hub portion having valving components operative to deliver insufflation gas to the patient, said hub portion being coupled to said tubular structure in a releasable manner.

15. The fluid delivery cannula of claim 12, further comprising a hub portion having valving components operative to deliver insufflation gas to the patient, said hub portion being formed integrally with said tubular structure.

16. The fluid delivery cannula of claim 15, wherein said hub portion and said tubular structure are integrally molded from a polymeric material.

17. The fluid delivery cannula of claim 12, wherein tubular structure further comprises at least two separate layers include an inner rigid tubular member and an outer sheath carried on said inner rigid tubular member, said inner rigid tubular member including a grooved surface for providing said fluid passage and said outer sheath operative to seal said fluid passage against leakage.

18. The fluid delivery cannula of claim 17, wherein said outer sheath is comprised of a polymeric material carried on said grooved surface.

19. The fluid delivery cannula of claim 18, wherein said polymeric material includes PTFE.

20. The fluid delivery cannula of claim 18, wherein said outer sheath is heat shrunk onto said grooved outer surface.

21. The fluid delivery cannula of claim 12, wherein said tubular structure is radially expandable.

22. A fluid delivery cannula for use in minimally invasive surgical procedures performed through a port site of a patient, comprising:
a radially expandable tubular structure including a central lumen configured to receive a trocar and an outer surface adapted to interface with tissue at the port site, said tubular structure further including a distal end and at least one fluid passage having an inlet and an outlet, said outlet communicating with at least one of said outer surface and said distal end for delivering fluid thereto.

23. A method of performing a minimally invasive surgical procedure using a trocar-cannula complex with a cannula portion having a lumen configured to receive a trocar and a separate fluid passage configured to receive fluid from an inlet on the trocar-cannula complex and deliver the fluid to an outlet on an outside surface of the cannula portion, the method comprising:
introducing the trocar-cannula complex through a port site of a patient,
coupling the inlet of the fluid passage to a source of fluid, and
delivering the fluid from the inlet through the fluid passage and the outlet on the outside surface of the cannula portion into contact with the patient.

24. The method of claim 23, wherein the fluid is an irrigant.

25. The method of claim 23, wherein the fluid includes a pain medication.

26. The method of claim 23, wherein the fluid is a tissue adhesive.

27. The method of claim 23, wherein the fluid is delivered to the inlet by a pump.

28. The method of claim 23, wherein the fluid is delivered to the inlet by a syringe.

29. A method of performing a minimally invasive surgical procedure using a trocar and a cannula having at least a portion that is radially expandable, the cannula further including a distal end, an outside surface, a lumen configured to receive the trocar, and a fluid passage extending from an inlet to an outlet on at least one of the outside surface and the distal end, the method comprising:
introducing the cannula and trocar through a port site of a patient,
coupling the inlet of the fluid passage to a source of fluid, and
delivering the fluid from the inlet through the fluid passage to the outlet and into contact with the patient.

30. The method of claim 29, wherein the step of introducing a cannula through a port site further comprises:
introducing a radially expandable cannula through the port site.

31. The method of claim 30, wherein the step of introducing a radially expandable cannula through a port site further comprises:
using a needle introducer with the radially expandable cannula positioned on the outside thereof,
withdrawing the needle introducer from the radially expandable cannula leaving the radially expandable cannula in the port site, and
inserting the trocar through the radially expandable cannula and the port site.
